(19) United States
(12) Patent Application Publication
(10) Pub. No.: US 2026/0151319 A1
(43) Pub. Date: Jun. 4, 2026

(54) Retinol Staging

(71) Applicant: Azadeh Shirazi, La Jolla, CA (US)

(72) Inventor: Azadeh Shirazi, La Jolla, CA (US)

(21) Appl. No.: 18/968,532

(22) Filed: Dec. 4, 2024

(51) Int. Cl. A61K 8/67; A61K 8/42; A61K 8/73; A61Q 19/00

(52) U.S. Cl. CPC A61K 8/671; A61K 8/42; A61K 8/735; A61Q 19/007

(57) ABSTRACT

A method for improving the effectiveness and reducing the irritation of retinol application by introducing a two-step evening skincare regimen, referred to as “Retinol Staging.” The method includes cleansing the skin and applying a moisturizer in the early evening to enhance hydration and barrier function, followed by a waiting period to allow absorption. Retinol is then applied at bedtime to maximize efficacy and minimize side effects. This approach differs from existing methods like “retinol sandwiching” and “skin cycling,” offering superior hydration and skin barrier protection.

### BACKGROUND OF THE INVENTION

### FIELD

The present application relates to methods for topical retinol application, particularly a staged application method to enhance absorption, improve hydration, strengthen the skin barrier, and minimize irritation and side effects.

### BACKGROUND

Topical retinol is a widely used ingredient in dermatology for its anti-aging and acne-treating properties. However, common application techniques, such as the “retinol sandwich” method, often lead to compromised absorption and irritation due to insufficient hydration or lack of proper timing. Similarly, “skin cycling” methods alternate retinol application with rest days, which may limit consistent efficacy.

### SUMMARY

The present invention introduces a novel “Retinol Staging” method that incorporates an early hydration step followed by a waiting period before bedtime application of retinol. This method optimizes skin hydration, barrier function, and retinol absorption, resulting in enhanced benefits and reduced side effects.

### DETAILED DESCRIPTION

In the following detailed description, the method, referred to as “Retinol Staging,” comprises the following steps:

### 1. Early Evening Cleansing and Moisturizing

The skin is cleansed using a gentle cleanser to remove impurities and prepare the skin.
A barrier-strengthening moisturizer is applied immediately after cleansing to replenish hydration.

### 2. Rest Period

After moisturizing, the skin is left undisturbed for at least one hour, allowing hydration to penetrate and the barrier to stabilize.

### 3. Bedtime Retinol Application

A topical retinol formulation is applied at bedtime.
The pre-moisturized and hydrated skin ensures optimal retinol absorption and reduces the risk of irritation and dryness commonly associated with retinol use.

This method is particularly advantageous for individuals with sensitive skin or those experiencing irritation from retinol use. By incorporating the hydration step and waiting period, the skin barrier is better equipped to handle the active ingredient, leading to improved tolerance and results.

### Advantages Over Prior Art

Unlike the “retinol sandwich” method, which applies moisturizer before and after retinol, or “skin cycling,” which spaces out retinol application, this method achieves:
1. Maximum hydration without diluting retinol.
2. Consistent retinol efficacy without rest days.
3. Reduced irritation and side effects due to a stabilized barrier.

### EXAMPLES OF USE

Example 1: A 35-year-old woman with combination skin follows the Retinol Staging method using a gentle cleanser, a hyaluronic acid-based moisturizer, and a 0.5% retinol formulation. Over 8 weeks, she observes significant improvements in skin texture and tone with minimal dryness or irritation.

Example 2: A 45-year-old man with sensitive skin implements the Retinol Staging method with a ceramide-rich moisturizer and a low-strength retinol serum. The method enhances his skin's tolerance to retinol while reducing redness and flakiness.

1. A method for enhancing the efficacy and minimizing the side effects of topical retinol application, comprising:
cleansing the skin and applying a moisturizer during an early evening period, wherein said moisturizer strengthens the skin barrier and enhances hydration;
allowing the moisturizer to absorb and the skin to achieve a hydrated state during a rest period of at least one hour; and
applying a topical retinol formulation to the skin at bedtime, wherein the application occurs after said rest period to optimize retinol absorption while reducing skin irritation.
